(19) United States
(12) Patent Application Publication
(10) Pub. No.: US 2021/0106579 A1
(43) Pub. Date: Apr. 15, 2021

(54) AMELIORATION OF COGNITIVE AND MOTOR DEFICITS ASSOCIATED WITH ALZHEIMER'S

(71) Applicant: Neuralstem, Inc., Germantown, MD (US)

(72) Inventor: Karl K. JOHE, Doral, FL (US)

(73) Assignee: Neuralstem, Inc., Germantown, MD (US)

(21) Appl. No.: 17/055,545

(22) PCT Filed: May 17, 2019
(86) PCT No.: PCT/US2019/032871

§ 371 Date: Nov. 13, 2020

Related U.S. Application Data
(60) Provisional application No. 62/673,004, filed on May 17, 2018.

(51) Int. Cl. A61K 31/496; A61P 25/28

(52) U.S. Cl. CPC A61K 31/496; A61P 25/28

(57) ABSTRACT

Cognitive and/or motor deficiencies associated with Alzheimer's are treated using 2-amino substituted nicotinamides on their pharmaceutically acceptable salts.

### CROSS-REFERENCE TO RELATED APPLICATIONS

This application claims priority of U.S. Provisional Application No. 62/673,004, filed 17 May 2018, the disclosures of which are herein incorporated by reference herein in its entirety.

### TECHNICAL FIELD

The invention relates to treatment of Alzheimer subjects with compounds that ameliorate the cognitive and motor deficits associated with this condition. More particularly, it concerns the use of 2-amino substituted nicotinamides for this purpose.

### BACKGROUND ART

Alzheimer's disease is an increasingly prevalent condition in developed countries and contributes significantly to the cost of health care. Cognitive defects associated with this condition are of the greatest concerns.

A family of U.S. granted patents, represented by, for example, U.S. Pat. No. 8,362,262, discloses low molecular weight compounds that are capable of stimulating neuronal growth. Subsequently, it was found that certain 2-amino-substituted nicotinamides were useful in treating depression, in particular, major depressive disorder in humans as described in PCT publication WO2015/195567. It was later found that the cognitive deficits experienced as a result of diabetes, stroke, Angelman's Syndrome or radiation could be remedied or ameliorated by these compounds as described in PCT/US 2017/050312; PCT/US 2018/018014; PCT/US 2017/061187 and PCT/US 2017/057233. The cognitive deficits associated with Alzheimer's, however, are not addressed by these documents.

### DISCLOSURE OF THE INVENTION

It has now been found that certain 2-amino-substituted nicotinamides are especially useful in prevention or reduction of cognitive and motor deficits associated with Alzheimer's disease.

Accordingly, in one aspect, the invention is directed to a method to ameliorate the cognitive or motor deficiencies associated with Alzheimer's by administering to a subject in need of such amelioration, a pharmaceutical composition wherein the active ingredient is a 2-amino-substituted nicotinamide or a pharmaceutically acceptable salt thereof. In particular, the 2-amino-substituted nicotinamide is of the formula:

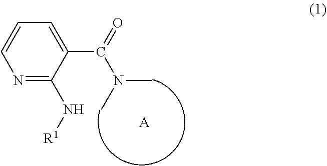

wherein R¹ is an alkyl of 3-8C and ring A is a 5- or 6-membered saturated ring optionally including an additional nitrogen which is unsubstituted or substituted with an additional nitrogen-containing substituent or a ring-opened form thereof.

Particular exemplified compounds include those of formula (2)

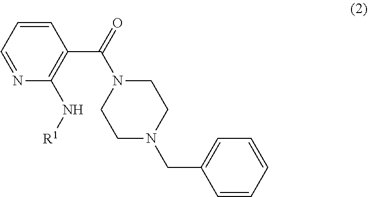

or formula (3)

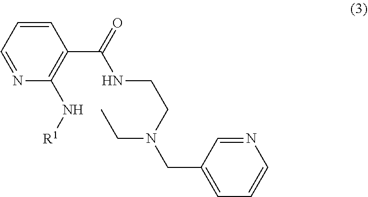

wherein R¹ is a branched alkyl group of 3-5C or of formula (4)

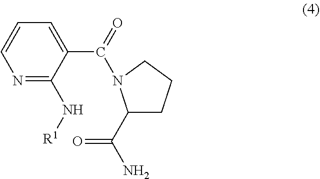

wherein R¹ is an alkyl group comprising a 5- or 6-membered ring.

The invention also includes pharmaceutical compositions and pharmaceutically acceptable salts of the compounds of the invention, in particular phosphate salts thereof.

### BRIEF DESCRIPTION OF THE DRAWINGS

FIG. 1 shows results of learning as measured by the Barnes maze test after six weeks of administration of vehicle or NSI-189.

FIG. 2 shows the results on memory retention as measured by the Barnes maze test after six weeks of administration of the medicament.

FIG. 3 shows the results of learning as measured by the Barnes maze test after 12 weeks of administration.

FIG. 4 shows the results on memory retention after 12 weeks of administration.

FIG. 5 shows the effect of administering NSI-189 for six weeks on short term memory as measured by the object recognition test.

FIG. 6 shows the results as in FIG. 5, but after 12 weeks of administration.

FIG. 7 shows the results of motor skill testing on a Rota-Rod system after six weeks of administration.

FIG. 8 shows the results of 6 and 12 weeks of administration of NSI-189 or vehicle on anxiety.

### MODES OF CARRYING OUT THE INVENTION

The methods of the invention are directed to ameliorating the cognitive and/or motor deficiencies associated with Alzheimer's disease. Cognitive and motor deficiencies appear, of course, in everyday life, but they can be measured by a number of procedures well known in the art. In human subjects, a particularly useful diagnostic is measurement by CogScreen, a computer-administered cognitive test battery required by the U.S. Federal Aviation Administration (FAA) for evaluation of the neurocognitive functioning of pilots and which has also played a key role in the FDA drug approval and labeling process (CogScreen LLC, St Petersburg, Fla.). This includes analysis of Shifting Attention Test-Arrow Color Accuracy a measure of executive functioning; Shifting Attention Test-Arrow Direction Reaction Time Correct, a measure of attention; Symbol Digit Coding-Delayed Recall Accuracy, a measure of memory and Shifting Attention Test-Instruction Number Incorrect, which is a measure of working memory. One or a combination of these aspects or a subset thereof may be employed.

Motor skills can be measured by the Bruininks Motor Ability Test (BMAT) or a battery of motor skills tests found on the web at ukk-instituutti or any of the number of recognized tests.

Among these used in laboratory models are Barnes maze performance which tests learning and memory, an object recognition test which tests memory, a learning Rota-Rod test that measures motor skills and additional tests that are known in the art such as those described by Jolivalt, C. G., et al., Exp. Neurol. (2010) 422-431 and King, M. R., et al., J. Neurosc. Res. (2013) 91:506-514.

Many tests appropriate for a particular type of subject are known in the art and any of these may be used in the context of the present invention.

The active agents useful in the method of the invention have the general formula (1) noted above wherein R¹ is an alkyl of 3-8C and ring A is a 5- or 6-membered saturated ring optionally including an additional nitrogen or a ring-opened form thereof. Thus, R¹ may be, in formula (1), a straight or branched chain alkyl group of at least 3C, such as isopropyl, secondary butyl, n-butyl, isoamyl, sec-amyl, hexyl, isohexyl and the like or comprise a saturated ring. Preferably in formula (2) or (3), R¹ is a branched alkyl of 3-5C, in particular isoamyl and, in formula (4), R¹ comprises a 5- or 6-membered saturated ring. Preferred embodiments of ring A are a piperidine or piperazine ring or ring opened forms thereof or a pyrrolidine ring. Typically, ring A is substituted with at least an additional nitrogen-containing substituent, including a substituent including an additional pyridine ring such as pyridyl methyl, or pyridyl ethyl or is a simpler substituent such as a carboxamide. Preferred forms of ring A are shown in formulas (2), (3) and (4) above along with appropriate substituents.

In some embodiments the compounds of Formula (2) or (3) are employed, especially wherein R¹ is isoamyl.

The compounds of the invention are formulated in standard pharmaceutical formulations such as those found in Remington's Pharmaceutical Sciences, latest edition, Mack Publishing Co., Easton, Pa. and include formulations for oral administration and parenteral administration. Typically, the compounds are administered orally in the form of tablets, capsules or in formulations that are administered as syrups or any other standard formulation. In some instances, the formulations may be designed for delayed release or may be designed for more instantaneous delivery. Parenteral administration may also be used. A variety of formulations that would be suitable for the compounds of the invention is known in the art and is subject to the decision of the practitioner with regard to route of administration.

Dosage levels also depend on the judgment of the practitioner, but are generally in the range of 0.01 mg/kg to 1-2 g/kg.

In general, the subjects of the treatment will be humans, although it is useful to employ laboratory animals as well in order to assess appropriate dosages, routes of administration and formulations. Thus, the subjects of the invention include not only humans, but laboratory research animals such as rabbits, rats, mice and the like. In some instances, other mammalian subjects may be appropriate such as in veterinary contexts where the subject may be ovine, bovine or equine or the subject may be a companion animal such as dog or cat.

The compounds of the invention may be administered in the form of their pharmaceutically acceptable salts such as halides, maleates, succinates, nitrates and the like. Particularly favored are phosphate salts.

The frequency of administration and dosage schedules are also dependent on the practitioner and the dose may be chronic and on a daily basis, weekly basis or more frequent, or a single dosage may suffice. The compounds of the invention may also be administered in combination with other active agents either in the same composition or sequentially.

The following examples illustrate, but do not limit the invention,

### EXAMPLE 1

### Effect of Test Compounds on an Alzheimer's Model

A mouse model of Alzheimer's disease obtained from Jackson Laboratory was used in this study. The model is B6SJLF1 with mutant APP and mutant PS1 transgenes (designated 5XFAD) exhibits Alzheimer's symptoms. Wild type control B6SJLF1/J mice were used. 5XFAD mice developed cognitive impairments at 4-5 months of age. Four groups of mice were employed, 15 mice per group, a wild type control administered vehicle; a wild type control administered NSI-189 as the phosphate salt administered orally at a level of 30 mg/kg per day and 5XFAD mice administered vehicle or administered NSI-189 as the phosphate salt orally also at 30 mg/kg/day (of the organic moiety). NSI-189 is of Formula 2 wherein R¹ is isoamyl. The treatment began at 15 weeks and was maintained for 12 weeks with measurements made after 6 weeks and 12 weeks.

### Barnes Maze

The Barnes maze test is described in King, M. R., et al., J. Neurosc. Res. (supra). The Barnes maze consists of a brightly lit, circular, white platform with 20 holes with equidistant spacing around the periphery. An escape box is placed beneath one of the holes marked by a visual clue. Before testing begins, the mouse is placed in the center of the platform and allowed to explore until either it finds the escape box or five minutes has elapsed. The time to locate the escape box is then recorded at the end of the session, the mouse is left in, or placed in, the escape box for an additional minute.

This system is used to test both learning and memory. To test learning, the mouse was placed in the maze for five consecutive days to ascertain whether the time required to find the escape box was decreased, which would indicate learning. To test memory retention, a subsequent period of two days was allowed to elapse and the mouse was retested to ascertain whether a decreased time in finding the escape box was found.

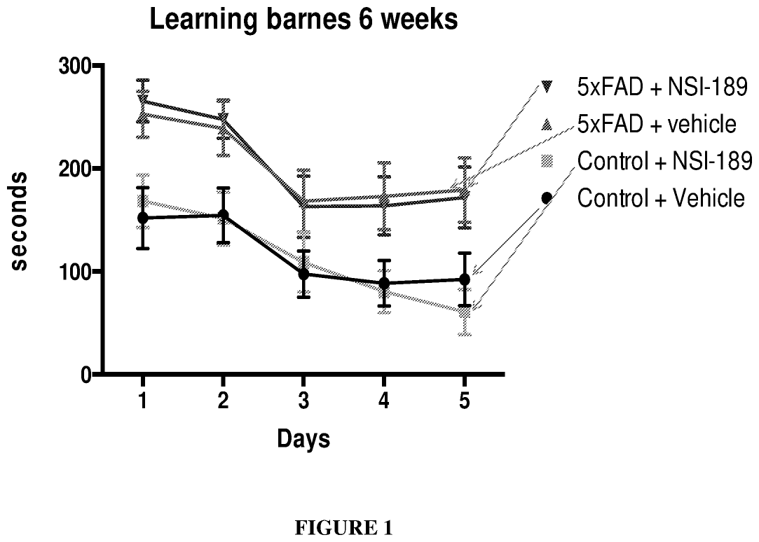

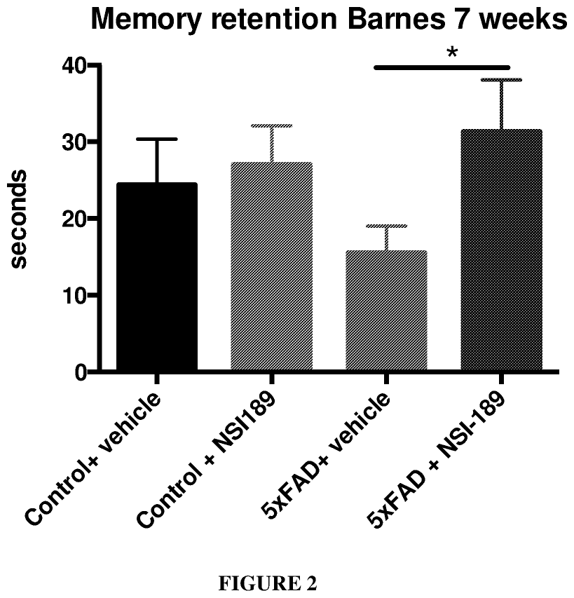

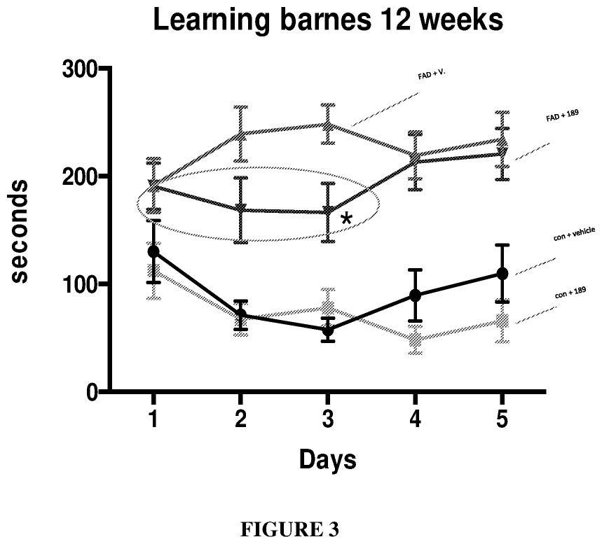

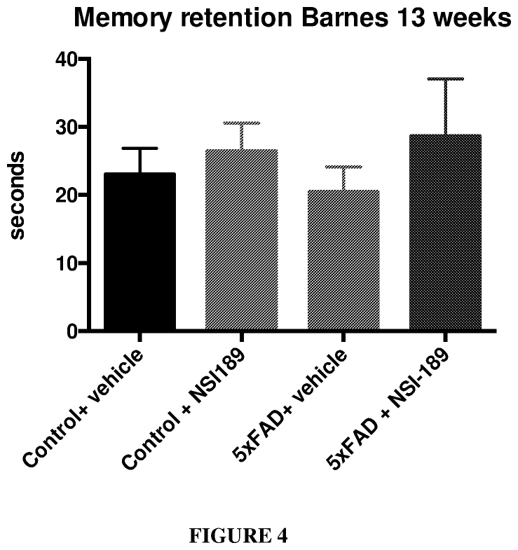

The results at six weeks for learning and memory are shown in FIGS. 1 and 2. As shown in FIG. 1, the controls are better at learning than the 5XFAD mice, and the administration of NSI-189 appeared not to affect the learning curve of either group. However, upon administering the test for memory retention, administration of NSI-189 to the 5XFAD mice improved memory (FIG. 2). As shown in FIGS. 3 and 4, 12 weeks of treatment with NSI-189 resulted in amelioration of worsened learning deficit, as well as in improvement of memory retention.

In summary six weeks of daily treatment with NSI-189 did not affect the learning ability of 5XFAD mice, but 12 weeks of treatment with NSI-189 ameliorated the learning disability of 7 month old 5XFAD mice at least at first. In the Barnes maze test, memory retention after two days was significantly improved by 6 weeks of treatment with NSI-189 in the 5XFAD mice as well as after 12 weeks of said treatment.

### Object Recognition

The object recognition test is also described in King, M. R., et al. (supra). This is based on a concept that the mice will explore a novel object for a longer time than a familiar object. Two identical objects are placed equal distance from each other and from the side of a transparent box and mice are given 10 minutes to achieve 30 seconds of exploration of the two objects after which they are placed back in their home cage. Exploration is defined as the mouse facing and being within approximately one inch of the object. An hour later, one of the objects is replaced with a novel object and the mouse is given five minutes to explore. The time that the mouse spends exploring the familiar and novel object is recorded and mice with normal cognitive function typically spend more time with a novel object.

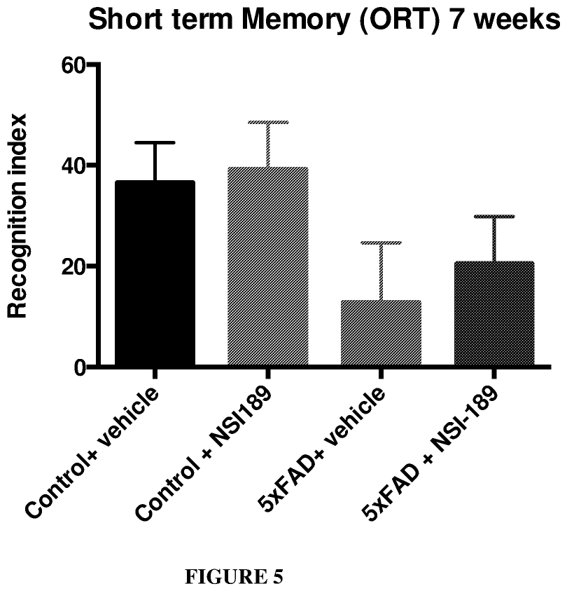

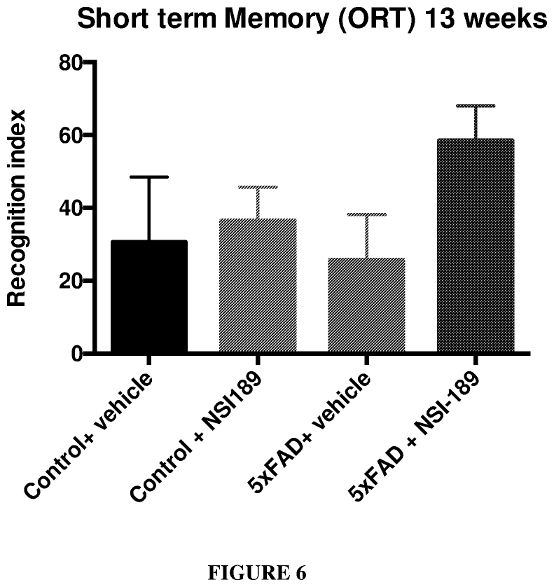

The results of this test are shown in FIGS. 5 and 6. As shown in FIG. 5, administration of NSI-189 improved the recognition index of 5XFAD mice as did administration over 12 weeks (FIG. 6). In fact, the recognition index of these mice exceeded that of controls.

### Learning Rota-Rod

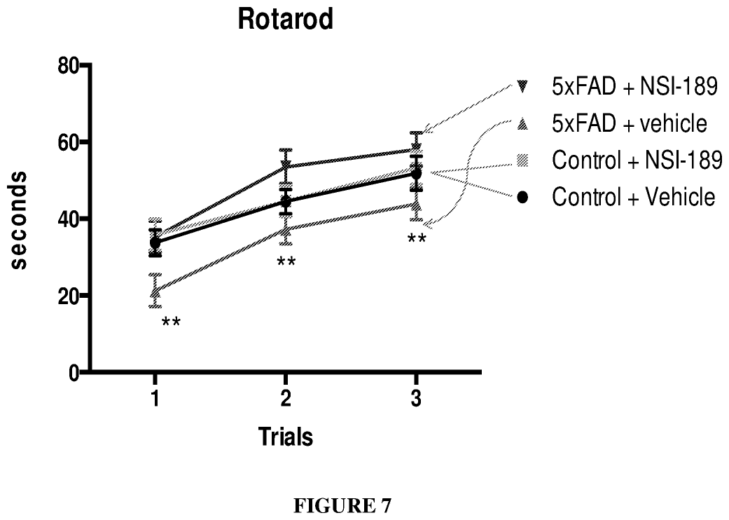

The Rota-Rod test also is described in King, et al. (supra). Rota-Rod is marketed by Stoelting Company, Wood Dale, Ill. This is a rod that rotates accelerating from 4-40 rpm over five minutes and the amount of time spent on the rod before loss of balance is recorded over three trials. This measures both motor skills, and if conducted over a period of days, it measures learning. As shown in FIG. 7, both the initial level of motor skills and the success in learning is decreased in 5XFAD mice, but administration of NSI-189 to these mice improves both the motor skill exhibited at the beginning and learning even over control.

In no case did NSI-189 affect the behavior of control mice.

### Anxiety

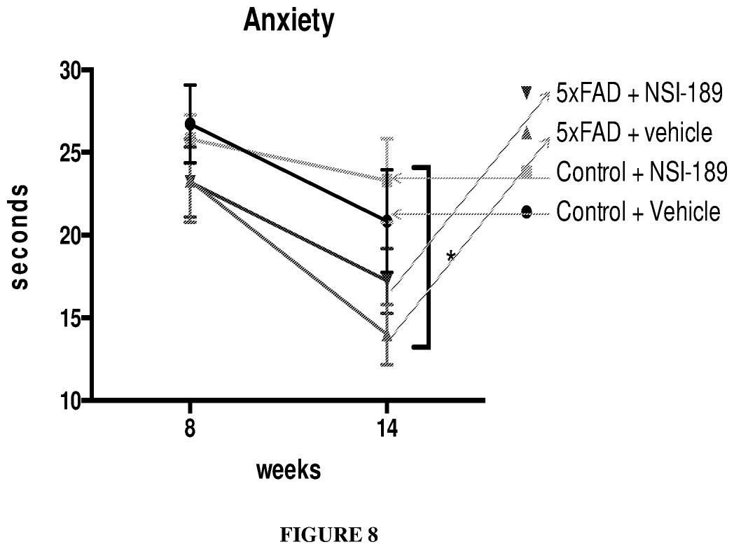

The time spent on the edge of an inverted beaker is used to assess anxiety, and the lower the time the more the anxious the mouse. At five months both control and 5XFAD mice had similar behaviors, and treatment with NSI-189 did not affect either group. At 7 months, however, the anxiety levels are increased for control and 5XFAD mice, but six additional treatment weeks with NSI-189 partially reduced the anxiety in both, as shown in FIG. 8.

1. A method to ameliorate cognitive and/or motor deficiencies associated with Alzheimer's disease which method comprises administering to a subject in need thereof a 2-amino substituted nicotinamide or a pharmaceutically acceptable salt thereof.

2. The method of claim 1 wherein cognitive skills are tested by the Barnes maze and/or object recognition tests and motor skills by the Rota-Rod test, where the subject is a laboratory model.

3. The method of claim 1 wherein cognitive skills are tested by CogScreen and motor skills by BMAT when the subject is human.

4. The method of claim 1 wherein the 2-amino substituted nicotinamide is NSI-189 of the formula: 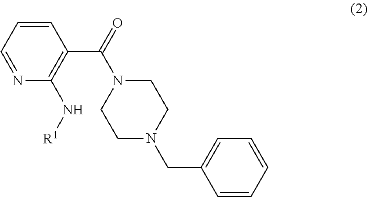
wherein R¹ is isoamyl.

5. The method of claim 1 wherein pharmaceutically acceptable salt is a phosphate salt.

6. The method of any of claims 1-5 which further includes subsequent testing of said subject for amelioration of said cognitive or motor skills deficiency.

7. The method of claim 6 wherein said cognitive testing comprises a Barnes maze test, and/or an object recognition test and the motor skills testing comprises a Rota-Rod test, when the subject is a laboratory model.

8. The method of claim 6 wherein cognitive skills are tested by CogScreen and motor skills by BMAT, when the subject is human
